(19) United States
(12) Patent Application Publication
(10) Pub. No.: US 2024/0228479 A1
(43) Pub. Date: Jul. 11, 2024

(54) PROCESS FOR SYNTHESIZING 2-BROMOLYSERGIC ACID DIETHYLAMIDE VIA CONTROLLED BROMINATION OF LYSERGIC ACID

(71) Applicant: Ceruvia Lifesciences LLC, Greenwich, CT (US)

(72) Inventors: Chris Atherton, Chester-le-Street (GB); Derek Londesbrough, Hartlepool (GB)

(73) Assignee: Ceruvia Lifesciences LLC, Greenwich, CT (US)

(21) Appl. No.: 18/400,102

(22) Filed: Dec. 29, 2023

Related U.S. Application Data
(60) Provisional application No. 63/436,514, filed on Dec. 31, 2022.

(51) Int. Cl. C07D 457/00; A61K 31/48

(52) U.S. Cl. CPC C07D 457/00; A61K 31/48

(57) ABSTRACT

Disclosed herein, inter alia, are a process for the preparation of 2-bromolysergic acid diethylamide (2-Br-LSD), or a pharmaceutically acceptable salt thereof, via the controlled bromination of lysergic acid to form 2-bromolysergic acid, followed by amidation to form 2-Br-LSD, the purified 2-Br-LSD, per se, and pharmaceutical compositions containing the purified 2-Br-LSD, per se, and uses thereof.

### RELATED APPLICATIONS

This application claims the benefit of priority under 35 U.S.C. § 119(e) to U.S. Provisional Application No. 63/436,514, filed Dec. 31, 2022, which is incorporated herein by reference in its entirety.

### BACKGROUND OF THE DISCLOSURE

2-Bromolysergic acid diethylamide (2-Br-LSD) is under investigation for a variety of pharmacological uses, including preventing and treating disorders associated with cephalic pain. The most efficient known synthetic routes for making 2-Br-LSD involve the reaction of lysergic acid diethylamide (LSD) with a brominating agent such as N-bromosuccinimide (NBS). However, variation of the brominating reagents, solvents, catalysts, and temperatures, provides, at best, a mixture of 2-Br-LSD and various di- and tri-brominated species. These di- and tri-brominated species have proven difficult to separate from 2-Br-LSD by readily scalable methods such as extraction, trituration, crystallization, or chromatography. The di- and tri-bromo impurities have been identified as having one bromine at the desired 2-position and the other bromine(s) on the phenyl ring of the indole moiety at positions 12, 13 and/or 14 (ergoline numbering).

### SUMMARY OF THE DISCLOSURE

A first aspect of the present disclosure is directed to a method of synthesizing pharmaceutical grade 2-bromolysergic acid diethylamide (2-Br-LSD), or a pharmaceutically acceptable salt thereof, comprising: (a) contacting lysergic acid with bromotrimethylsilane (TMSBr) and dimethylsulfoxide (DMSO), wherein the TMSBr and DMSO are in molar equivalents of about 6 and about 0.9, respectively, to form 2-bromolysergic acid; (b) contacting the 2-bromolysergic acid with diethylamine and an amide coupling reagent, to form 2-Br-LSD; (c) filtering a mixture of the 2-Br-LSD and a solvent; (d) allowing the 2-Br-LSD to precipitate from the filtered mixture; (e) collecting the 2-Br-LSD by filtration; (f) analyzing the collected 2-Br-LSD of (e) for the presence of iso-2-Br-LSD; and (g1) if a batch of the collected 2-Br-LSD of (e) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or (g2) if a batch of the collected 2-Br-LSD of (e) fails to meet the pre-set specification for iso-2-Br-LSD, then purifying the collected 2-Br-LSD and repeating (f) and (g), or discarding the collected 2-Br-LSD.

Another aspect of the present disclosure provides a batch of pharmaceutical grade 2-Br-LSD, or a pharmaceutically acceptable salt thereof, that meets pre-set specifications that comprise a pre-set specification for iso-2-Br-LSD, wherein the pre-set specification for iso-2-Br-LSD is not more than 0.4%.

A further aspect of the present disclosure provides a pharmaceutical composition, comprising a batch of the pharmaceutical grade 2-Br-LSD, or a portion thereof, and a pharmaceutically acceptable carrier.

The disclosed methods produce 2-Br-LSD in relatively high yield, at a high purity substantially free of impurities, particularly di- and tri-bromo species (having one bromine at the desired 2-position and the other bromine(s) on the phenyl ring of the indole moiety at positions 12, 13 and/or 14 (ergoline numbering)).

### BRIEF DESCRIPTION OF THE DRAWINGS

FIG. 1 is the X-ray powder (XRP) diffractogram of 2-bromo-LSD hemi-L-tartrate, designated as MK140H.

FIG. 2 is the differential scanning calorimetry (DSC) thermogram of 2-bromo-LSD hemi-L-tartrate, designated as MK140H.

### DETAILED DESCRIPTION OF THE DISCLOSURE

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 9%, 8%, 7%, 6%, 5%, 4%, 3%, 2%, 1%, or even less than 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the term “batch” refers to a specific quantity of a drug or other material (e.g., 2-bromolysergic acid diethylamide, 2-bromolysergic acid) that is intended to have uniform character and quality, within specified limits, and is produced according to a single manufacturing order during the same cycle of manufacture. See, 21 CFR Part 210, FDA, Guidance for Industry cGMP for Phase 1 Investigational Drugs, FDA.

As used herein, the terms “Good Manufacturing Practices” or “GMP,” “Current Good Manufacturing Practices” or “cGMP,” “Master Production Instructions,” “Batch Production Records,” “Quality Unit(s),” and “Active Pharmaceutical Ingredient” or “API,” follow The International Council for Harmonisation of Technical Requirements for Pharmaceuticals for Human Use (ICH) guidelines, which are a set of guidelines to ensure safe, effective and high-quality medicines are developed and registered efficiently. These guidelines have been adopted by regulatory authorities throughout the world, for example, “Q7 Good Manufacturing Practice Guidance for Active Pharmaceutical Ingredients,” (hereinafter “Q7”), which are incorporated herein by reference.

As used herein, the term “Temporary Processing Instructions” is used in the context of GMP to refer to a short-term change in batch processing instructions that is pre-approved by the manufacturer's Quality Unit(s).

As used herein, the term “pharmaceutical grade” refers to an Active Pharmaceutical Ingredient, which is manufactured according to Good Manufacturing Practices (GMP) as defined, for example, in Q7.

As used herein, the terms “precipitate,” “precipitation,” “crystallize” and “crystallization” are used interchangeably to refer to unit operations that generate a solid from a saturated solution. As these terms are used herein, no distinction is made as to whether said solid is amorphous or crystalline, has small particles or large particles, or precipitates rapidly or slowly.

As used herein, the term “chromatographic purification” refers to the separation of a mixture by passing it in solution or suspension or as a vapor (as in gas chromatography) through a medium in which the individual components of the mixture move at different rates. Some examples include column chromatography, flash column chromatography, high performance liquid chromatography, gas chromatography, affinity chromatography, supercritical fluid chromatography, and ion-exchange chromatography, among others known to persons skilled in the art.

As used herein, the term “ambient temperature” refers to the temperature of the immediate surroundings (e.g., room temperature or a reaction environment, e.g., about 20° C. to about 25° C.).

As used herein, the term “antisolvent” refers to a solvent in which a compound or salt thereof is poorly soluble and which induces precipitation or crystallization of the compound or salt thereof from solution.

As used herein, abbreviations for methods, techniques, limits, dimensionless quantities, and measured or calculated values may include High-Performance Liquid Chromatography (HPLC), Ultra Performance Liquid Chromatography (UPLC), Thin Layer Chromatography (TLC), Supercritical Fluid Chromatography (SCF), Capillary Electrophoresis (CE), Ultraviolet Absorbance Detection (UV), Gas Chromatography (GC), Mass Spectrometry (MS), Liquid Chromatography—Mass Spectrometry (LC-MS), Gas Chromatography—Mass Spectrometry (GC-MS), Nuclear Magnetic Resonance Spectroscopy (NMR), Fourier Transform Infrared Spectroscopy (FTIR), Good Manufacturing Practices (GMP), Current Good Manufacturing Practices (cGMP), Parts Per Million (ppm), Retention Time (RT), Relative Retention Time (RRT), Mass-To-Charge Ratio (m/z), Not More Than (NMT), and Not Less Than (NLT).

As used herein, % purity level and % impurity level refer to area %, unless indicated otherwise.

As used herein, the terms “2-bromolysergic acid diethylamide” and “2-Br-LSD” are used interchangeably and refer to (6aR,9R)-5-bromo-N,N-diethyl-7-methyl-4,6,6a,7,8,9-hexahydroindolo[4,3-fg]quinoline-9-carboxamide.

As used herein, the terms “iso-2-bromolysergic acid diethylamide” and “iso-2-Br-LSD” are used interchangeably and refer to (6aR,9S)-5-bromo-N,N-diethyl-7-methyl-4,6,6a, 7,8,9-hexahydroindolo[4,3-fg]quinoline-9-carboxamide.

As used herein, the term “lysergic acid” refers to (6aR,9R)-7-methyl-4,6,6a, 7,8,9-hexahydroindolo[4,3-fg]quinoline-9-carboxylic acid.

As used herein, the term “2-bromolysergic acid” refers to (6aR,9R)-5-bromo-7-methyl-4,6,6a, 7,8,9-hexahydroindolo[4,3-fg]quinoline-9-carboxylic acid.

As used herein, the term “iso-2-bromolysergic acid” refers to (6aR,9S)-5-bromo-7-methyl-4,6,6a, 7,8,9-hexahydroindolo[4,3-fg]quinoline-9-carboxylic acid.

As used herein, the terms “lysergic acid diethylamide” and “LSD” are used interchangeably and refer to (6aR,9R)—N,N-diethyl-7-methyl-4,6,6a,7,8,9-hexahydroindolo[4,3-fg]quinoline-9-carboxamide.

As used herein, the terms “iso-lysergic acid diethylamide” and “iso-LSD” are used interchangeably and refer to (6aR,9S)—N,N-diethyl-7-methyl-4,6,6a,7,8,9-hexahydroindolo[4,3-fg]quinoline-9-carboxamide.

As used herein, the term “2,12-dibromo-lysergic acid” refers to (6aR,9R)-1,5-dibromo-7-methyl-4,6,6a, 7,8,9-hexahydroindolo[4,3-fg]quinoline-9-carboxylic acid.

As used herein, the term “2,13-dibromo-lysergic acid” refers to (6aR,9R)-2,5-dibromo-7-methyl-4,6,6a, 7,8,9-hexahydroindolo[4,3-fg]quinoline-9-carboxylic acid.

As used herein, the term “2,14-dibromo-lysergic acid” refers to (6aR,9R)-3,5-dibromo-7-methyl-4,6,6a, 7,8,9-hexahydroindolo[4,3-fg]quinoline-9-carboxylic acid.

As used herein, the term “2,12,13-tribromo-lysergic acid” refers to (6aR,9R)-1,2,5-tribromo-7-methyl-4,6,6a, 7,8,9-hexahydroindolo[4,3-fg]quinoline-9-carboxylic acid.

As used herein, the term “2,12,14-tribromo-lysergic acid” refers to (6aR,9R)-1,3,5-tribromo-7-methyl-4,6,6a, 7,8,9-hexahydroindolo[4,3-fg]quinoline-9-carboxylic acid.

As used herein, the term “2,13,14-tribromo-lysergic acid” refers to (6aR,9R)-2,3,5-tribromo-7-methyl-4,6,6a, 7,8,9-hexahydroindolo[4,3-fg]quinoline-9-carboxylic acid.

As used herein, the terms “2,12-dibromo-lysergic acid diethylamide” and “2,12-dibromo-LSD” are used interchangeably and refer to (6aR,9R)-1,5-dibromo-N,N-diethyl-7-methyl-4,6,6a, 7,8,9-hexahydroindolo[4,3-fg]quinoline-9-carboxamide.

As used herein, the terms “2,13-dibromo-lysergic acid diethylamide” and “2,13-dibromo-LSD” are used interchangeably and refer to (6aR,9R)-2,5-dibromo-N,N-diethyl-7-methyl-4,6,6a, 7,8,9-hexahydroindolo[4,3-fg]quinoline-9-carboxamide.

As used herein, the terms “2,14-dibromo-lysergic acid diethylamide” and “2,14-dibromo-LSD” are used interchangeably and refer to (6aR,9R)-3,5-dibromo-N,N-diethyl-7-methyl-4,6,6a, 7,8,9-hexahydroindolo[4,3-fg]quinoline-9-carboxamide.

As used herein, the terms “2,12,13-tribromo-lysergic acid diethylamide” and “2,12,13-tribromo-LSD” are used interchangeably and refer to (6aR,9R)-1,2,5-tribromo-N,N-diethyl-7-methyl-4,6,6a, 7,8,9-hexahydroindolo[4,3-fg]quinoline-9-carboxamide.

As used herein, the terms “2,12,14-tribromo-lysergic acid diethylamide” and “2,12,14-tribromo-LSD” are used interchangeably and refer to (6aR,9R)-1,3,5-tribromo-N,N-diethyl-7-methyl-4,6,6a, 7,8,9-hexahydroindolo[4,3-fg]quinoline-9-carboxamide.

As used herein, the terms “2,13,14-tribromo-lysergic acid diethylamide” and “2,13,14-tribromo-LSD” are used interchangeably and refer to (6aR,9R)-2,3,5-tribromo-N,N-diethyl-7-methyl-4,6,6a, 7,8,9-hexahydroindolo[4,3-fg]quinoline-9-carboxamide.

The structure of 2-Br-LSD is as follows:

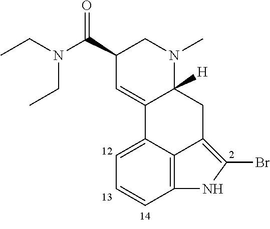 [SMILES: CCN(CC)C(=O)[C@@H]1C=C2c3cccc4[nH]c(Br)c(c34)C[C@H]2N(C)C1]

The structure of iso-2-Br-LSD is as follows:

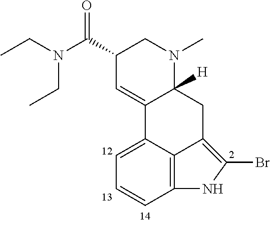 [SMILES: CCN(CC)C(=O)[C@H]1C=C2c3cccc4[nH]c(Br)c(c34)C[C@H]2N(C)C1]

The synthesis of 2-Br-LSD can result in a plurality of impurities following the end step synthesis. An illustrative synthesis of 2-Br-LSD, described in U.S. Pat. No. 10,377,752, entails bromination of methylergometrine with N-bromosuccinimide. The present inventors have discovered that synthesis of 2-Br-LSD via bromination of lysergic acid followed by amidation to form 2-Br-LSD, or by direct bromination of LSD, may result in formation of certain di- and tri-brominated impurities (having one bromine at the desired 2-position and the other bromine(s) on the phenyl ring of the indole moiety at positions 12, 13 and/or 14 (ergoline numbering)). The inventors have identified some of these impurities on the basis of mass spectrometric (MS) and nuclear magnetic resonance (NMR) spectroscopic data. These impurities include one or more of: 2,12-dibromo-lysergic acid diethylamide, 2,13-dibromo-lysergic acid diethylamide, 2,14-dibromo-lysergic acid diethylamide, 2,12,13-tribromo-lysergic acid diethylamide, 2,12,14-tribromo-lysergic acid diethylamide, 2,13,14-tribromo-lysergic acid diethylamide, iso-2-Br-LSD, LSD, and iso-LSD.

The inventors have also discovered that these impurities can be controlled and reduced to non-detectable or acceptably detectable levels. Any such impurity that occurs in an API intended for marketing approval at a level exceeding the relevant qualification threshold must either be reduced to a level below the qualification threshold or appropriately qualified. Qualification thresholds and methods for qualifying impurities are described in the relevant regulatory guidelines for impurities in new drug substances, such as ICH Q3A(R2) Impurities in New Drug Substances, 25 Oct. 2006 and ICH M7(R1) Assessment and Control of DNA Reactive (Mutagenic) Impurities in Pharmaceuticals to Limit Potential Carcinogenic Risk, 31 Mar. 2017. Qualification may include nonclinical and/or clinical studies, which can be expensive and time-consuming. Currently, the toxicological and pharmacological properties of 2,12-dibromo-lysergic acid diethylamide, 2,13-dibromo-lysergic acid diethylamide, 2,14-dibromo-lysergic acid diethylamide, 2,12,13-tribromo-lysergic acid diethylamide, 2,12,14-tribromo-lysergic acid diethylamide, 2,13,14-tribromo-lysergic acid diethylamide, iso-2-Br-LSD, LSD, and iso-LSD are generally not well understood. The most studied among these potential impurities is LSD, which is a potent hallucinogen. The identification of such impurities facilitates quality control and consistency of product. With the knowledge of the identities of these impurities, a manufacturer of 2-Br-LSD can set specifications for the maximum allowable amount of the impurities in bulk GMP 2-Br-LSD, which can then be formulated into bulk pharmaceutical composition and then distributed into pharmaceutical dosage units.

A first aspect of the present disclosure is directed to a method of synthesizing pharmaceutical grade 2-bromolysergic acid diethylamide (2-Br-LSD), or a pharmaceutically acceptable salt or thereof, comprising: (a) contacting lysergic acid with bromotrimethylsilane (TMSBr) and dimethylsulfoxide (DMSO), wherein the TMSBr and DMSO are in molar equivalents of about 6 and about 0.9, respectively, to form 2-bromolysergic acid; (b) contacting the 2-bromolysergic acid with diethylamine and an amide coupling reagent, to form 2-Br-LSD; (c) filtering a mixture of the 2-Br-LSD and a solvent; (d) allowing the 2-Br-LSD to precipitate from the filtered mixture; (e) collecting the 2-Br-LSD by filtration; (f) analyzing the collected 2-Br-LSD of (e) for the presence iso-2-Br-LSD; and (g1) if a batch of the collected 2-Br-LSD of (e) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or (g2) if a batch of the collected 2-Br-LSD of (e) fails to meet the pre-set specification for iso-2-Br-LSD, then purifying the collected 2-Br-LSD and repeating (f) and (g), or discarding the collected 2-Br-LSD.

The analysis of purity and impurity levels is determined by HPLC using UV detection (HPLC/UV) at 230 nm under the conditions described in Example 1, HPLC Method 4, and are provided as area %. HPLC Method 4 includes the following: System: Agilent 1100/1200 series liquid chromatograph or equivalent; Column: XSelect CSH C18; 150×4.6 mm, 3.5 μm particle size; Mobile phase A: Purified water:Acetonitrile:Trifluoroacetic acid (95:5:0.05%); Mobile phase A: Acetonitrile:Purified water:Trifluoroacetic acid (95:5:0.05%); Flow rate: 1.0 mL/min; Stop time: 21 min; Post run time: 5 min; Injection volume: 5 μL; Column temperature: 35° C.; Detection: UV 230 nm;

### Gradient:

| Time | % A | % B |
|---|---|---|
| 0 | 100 | 0 |
| 3 | 100 | 0 |
| 15 | 20 | 80 |
| 20 | 20 | 80 |
| 21 | 100 | 0 |

### Retention Times of Relevant Compounds:

| | | Relative | | |
|---|---|---|---|---|
| | Retention | retention | Concentration | |
| Compound | time (min) | time | of standard | Diluent |
| Lysergic acid | 8.27 | 0.80 | 0.30 mg/mL | methanol |
| 2-Bromolysergic | 9.11 | 0.88 | 0.30 mg/mL | methanol |
| acid | | | | |
| LSD | 9.85 | 0.95 | 0.30 mg/mL | methanol |
| Iso-LSD | 10.14 | 0.98 | 0.30 mg/mL | methanol |
| 2-Br-LSD | 10.37 | 1.00 | 0.30 mg/mL | methanol |
| Iso-2-Br-LSD | 10.83 | 1.04 | 0.30 mg/mL | methanol |

In some embodiments, steps (c)-(e) result in 2-Br-LSD that meets pre-set specifications that comprise a pre-set specification for iso-2-Br-LSD; thus, the collected 2-Br-LSD is accepted for further processing into pharmaceutical grade 2-Br-LSD, or a pharmaceutically acceptable salt thereof.

Persons skilled in the art may employ a number of methods for purifying the collected 2-Br-LSD of (e) in (g2), for example, extraction, trituration, crystallization, fractional crystallization, recrystallization, or chromatography. In some embodiments, the purifying comprises column chromatography. In some embodiments, the purifying comprises HPLC. In some embodiments, the purifying is performed in the absence of chromatographic purification.

In some embodiments, the purifying is performed by (al) suspending the collected 2-Br-LSD of (c) in ethyl acetate or isopropyl acetate, (b1) filtering the suspension, (c1) heating the filtrate to about 30-70° C., (dl) adding water in an amount less than about 3.5%, (el) allowing the mixture to cool, and (f1) collecting the product by filtration. In some embodiments, the purifying is performed by (al) suspending the collected 2-Br-LSD of (c) in ethyl acetate, (b1) filtering the suspension, (c1) heating the filtrate to about 45-50° C., (d1) adding water in an amount of about 2 molar equivalents relative to 2-Br-LSD, (el) allowing the mixture to cool, and (f1) collecting the product by filtration.

In some embodiments, the pre-set specification for iso-2-Br-LSD is not more than 2%. In some embodiments, the pre-set specification for iso-2-Br-LSD is not more than 0.4%. In some embodiments, the pre-set specification further comprises at least one of the following pre-set specifications: not more than 0.05% of LSD, not more than 0.5% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

In some embodiments, the pre-set specification further comprises at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

In some embodiments, (a) comprises contacting the lysergic acid with TMSBr and DMSO in the presence of a solvent or mixture of solvents. In some embodiments, the solvent is an organic solvent. Representative examples of organic solvents that may be suitable for use in the reaction include hydrocarbon solvents, including those containing one or more of the following elements: oxygen, nitrogen, sulfur, chlorine, fluorine. In some embodiments, the solvent is tetrahydrofuran (THF). Other organic solvents include 2-methyltetrahydrofuran, acetonitrile, dichloromethane, N,N-dimethylformamide, N-methylpyrrolidone, anisole, 1,2-dichloroethene, 1,2-dimethoxyethane, N,N-dimethylacetamide, and 1,4-dioxane.

In some embodiments, (a) comprises adding TMSBr to a pre-formed mixture containing lysergic acid, DMSO, and a solvent. In some embodiments, (a) comprises adding TMSBr to a mixture of DMSO and a solvent, followed by adding the resulting mixture to a pre-formed mixture containing lysergic acid and a solvent. In some embodiments, a pre-formed mixture of lysergic acid and a solvent is added to a pre-formed mixture of TMSBr, DMSO and a solvent. In some embodiments, a pre-formed mixture of lysergic acid, DMSO and a solvent is added to a pre-formed mixture of TMSBr and a solvent. In some embodiments, the addition of TMSBr to a mixture of lysergic acid, DMSO and a solvent is completed in not more than 5 minutes.

In some embodiments, about 4 to about 10 molar equivalents of TMSBr are used for each molar equivalent of lysergic acid. In some embodiments, about 5.5 to about 6.5 molar equivalents of TMSBr are used for each molar equivalent of lysergic acid. In some embodiments, about 6.0 molar equivalents of TMSBr are used for each molar equivalent of lysergic acid. In some embodiments, about 0.6 to about 1.0 molar equivalents of DMSO are used for each molar equivalent of lysergic acid. In some embodiments, about 0.8 to about 1.0 molar equivalents of DMSO are used for each molar equivalent of lysergic acid. In some embodiments, about 0.9 molar equivalents of DMSO are used for each molar equivalent of lysergic acid. In some embodiments, about 6.0 molar equivalents of TMSBr and about 0.9 molar equivalents of DMSO are used for each molar equivalent of lysergic acid.

In some embodiments, about 10 mL to about 100 mL of organic solvent is used per g of lysergic acid. In some embodiments, about 20 mL to about 30 mL of organic solvent is used per g of lysergic acid.

Lysergic acid, DMSO and the organic solvent may all be sources of water. In some embodiments, the water content of the reaction mixture is not more than about 10% weight-to-weight (w/w). In some embodiments, the water content of the reaction mixture is not more than about 5% weight-to-weight (w/w). In some embodiments, the water content of the reaction mixture is not more than about 2% weight-to-weight (w/w). In some embodiments, the water content of the reaction mixture is not more than about 1% w/w. In some embodiments, the water content of the reaction mixture is not more than about 0.2% w/w. In some embodiments, the water content of the lysergic acid is not more than about 4% w/w, and anhydrous THF and DMSO are used.

Selection of other reaction parameters such as temperature and time is within the level of skill in the art. In some embodiments, (a) is conducted at a temperature between about −30° C. and about 30° C. In some embodiments, (a) is conducted at a temperature between about −15° C. and about 10° C. In some embodiments, (a) is conducted at a temperature between about 0° C. and about 10° C. In some embodiments, (a) is conducted at a temperature between about −15° C. and about 0° C.

In some embodiments, (a) is conducted for about 5 min to about 60 min after all reactants have been combined. In some embodiments, (a) is conducted for about 30 min after all reactants have been combined.

In some embodiments, the method further comprises a step (a2) of isolating the 2-bromolysergic acid as a solid prior to conducting (b). In some embodiments, the isolating comprises the addition of a suitable anti-solvent to the reaction mixture of (a) and filtering the solid formed. Representative examples of anti-solvents include organic solvents (e.g., hydrocarbon solvents, including those containing one or more of the following elements: oxygen, nitrogen, chlorine, fluorine). In some embodiments, the anti-solvent is pentane, hexane, or heptane.

In some embodiments, (b) comprises contacting the 2-bromolysergic acid with diethylamine and an amide coupling reagent in the presence of a solvent or mixture of solvents. Representative examples of organic solvents that may be suitable for use in the reaction include hydrocarbon solvents, including those containing one or more of the following elements: oxygen, nitrogen, sulfur, chlorine, fluorine. In some embodiments, the solvent is tetrahydrofuran (THF). Other organic solvents include acetonitrile, dichloromethane, ethyl acetate, isopropyl acetate, toluene, and 2-methyltetrahydrofuran. In some embodiments, the solvent is dichloromethane. In some embodiments, the solvent is acetonitrile. In some embodiments, the solvent is a mixture of acetonitrile and dichloromethane.

Representative examples of amide coupling reagents that may be useful in the practice of the present disclosure include carbamates, carbazates, dicyclohexylcarbodiimide (DCC), benzotriazol-1-yloxytripyrrolidinophosphonium hexafluorophosphate (PyBOP), 2-(1H-benzotriazole-1-yl)-1,1,3,3-tetramethylaminium tetrafluoroborate (TBTU), 2-(1H-benzotriazol-1-yl)-1,1,3,3-tetramethyluronium hexafluorophosphate (hexafluorophosphate benzotriazole tetramethyl uronium, or HBTU), 1-[bis(dimethylamino)methylene]-1H-1,2,3-triazolo[4,5-b]pyridinium 3-oxide hexafluorophosphate (hexafluorophosphate azabenzotriazole tetramethyl uronium, or HATU), 1,1′-carbonyldiimidazole (CDI), propylphosphonic anhydride (T3P), N-(3-dimethylaminopropyl)-N′-ethylcarbodiimide (EDC) with or without hydroxybenzotriazole (HOBT), pivaloyl chloride, and acetic anhydride. In some embodiments, the amide coupling reagent is T3P. In some embodiments, the amide coupling reagent is HBTU.

In some embodiments, (b) comprises contacting 2-bromolysergic acid with diethylamine, an amide coupling reagent and a base. In some embodiments, (b) comprises contacting 2-bromolysergic acid with diethylamine, an amide coupling reagent and an acyl transfer promoter. In some embodiments, (b) comprises contacting 2-bromolysergic acid, diethylamine, an amide coupling reagent, a base and an acyl transfer promoter.

Representative examples of bases that may be useful in the practice of the present disclosure include triethylamine, N-methylpyrrolidine, N-methylmorpholine (NMM), and 1,4-diazo[2.2.2]octane (DABCO). Representative examples of acyl transfer promoters that may be useful in the practice of the present disclosure include pyridine, N-methylimidazole (NMI), and 4-(dimethylamino)pyridine (DMAP).

In some embodiments, about 1 to about 5 molar equivalents of the amide coupling agent are used for each molar equivalent of 2-bromolysergic acid. In some embodiments, about 1.2 to about 2.5 molar equivalents of the amide coupling agent are used for each molar equivalent of 2-bromolysergic acid. In some embodiments, about 1.1 to about 1.4 molar equivalents of the amide coupling agent are used for each molar equivalent of 2-bromolysergic acid. In some embodiments, about 1.8 to about 2.2 molar equivalents of the amide coupling agent are used for each molar equivalent of 2-bromolysergic acid.

In some embodiments, about 1 to about 10 molar equivalents of diethylamine are used for each molar equivalent of 2-bromolysergic acid. In some embodiments, about 4 to about 6 molar equivalents of diethylamine are used for each molar equivalent of 2-bromolysergic acid.

In some embodiments, neither a base (other than diethylamine) nor an acyl transfer promoter is used. In some embodiments, about 1 to about 10 molar equivalents of a base are used for each molar equivalent of 2-bromolysergic acid. In some embodiments, about 0.1 to about 10 molar equivalents of an acyl transfer promoter are used for each molar equivalent of 2-bromolysergic acid.

In some embodiments, about 1 to about 100 mL of organic solvent is used per g of 2-bromolysergic acid. In some embodiments, about 10 mL of organic solvent is used per g of 2-bromolysergic acid.

Selection of other reaction parameters such as temperature and time are within the level of skill in the art. In some embodiments, the amide coupling is conducted at a temperature between about −20° C. and about 35° C. In some embodiments, the amide coupling is conducted at a temperature between about 0° C. and about 25° C. In some embodiments, the amide coupling is conducted at a temperature between about 0° C. and about 5° C. In some embodiments, the amide coupling is conducted at a temperature between about 15° C. and about 25° C.

In some embodiments, the amide coupling is conducted for about 15 min to about 24 h. In some embodiments, the amide coupling is conducted for about 30 min to about 2 h. In some embodiments, the amide coupling is conducted until in-process analysis shows complete consumption of 2-bromolysergic acid. In some embodiments, in-process analysis is performed by HPLC at a detection wavelength of about 230 nm. In some embodiments, in-process analysis is performed by TLC. If in-process analysis indicates that substantial 2-bromolysergic acid remains, additional diethylamine may be added to drive the reaction to completion.

In some embodiments, the amide coupling comprises an aqueous workup. In some embodiments, the aqueous workup comprises washing with an aqueous base. Representative examples of aqueous bases that may be useful include potassium carbonate, potassium bicarbonate, sodium carbonate, sodium bicarbonate, sodium hydroxide, and potassium hydroxide. In some embodiments, the aqueous base is aqueous potassium carbonate.

In some embodiments, the 2-Br-LSD may be present substantially in a solution, and filtration of the solution is performed to remove undesired impurities. In some embodiments, the 2-Br-LSD may be allowed to precipitate or crystallize from the filtered mixture, or filtrate, using standard precipitation or crystallization techniques, known to persons skilled in the art. In some embodiments, precipitation or crystallization may be induced by allowing the filtrate to cool. In some embodiments, precipitation or crystallization may be induced by allowing the filtrate to cool from a temperature above about 20° C. to about 25° C. to a temperature about 20° C. to about 25° C. In some embodiments, precipitation or crystallization may be induced by cooling the filtrate to a temperature below ambient. In some embodiments, precipitation or crystallization may be induced by allowing the filtrate to cool to about 0-5° C. In some embodiments, precipitation or crystallization may be induced by slow evaporation of the filtrate. In some embodiments, precipitation or crystallization may be induced by the addition of an antisolvent to the filtrate. In some embodiments, the antisolvent is water. In some embodiments, the antisolvent is water in an amount that does not exceed the maximum solubility of water in the filtrate. In some embodiments, the antisolvent is water in an amount less than about 3.5%. In some embodiments, the antisolvent is water in an amount of about 2 molar equivalents relative to 2-Br-LSD. In some embodiments, precipitation or crystallization may be induced by allowing the vapor of an antisolvent to diffuse into the filtrate.

In some embodiments, the solvent of (c) comprises an organic solvent. In some embodiments, the organic solvent comprises one or more of isopropyl acetate, ethyl acetate, tert-butyl methyl ether, toluene, cyclopropyl methyl ether, butyl acetate, isobutyl acetate, methyl acetate, propyl acetate, methyl ethyl ketone, methyl isobutyl ketone, acetone, and acetonitrile. In some embodiments, the organic solvent comprises isopropyl acetate, ethyl acetate, tert-butyl methyl ether, toluene, or cyclopropyl methyl ether. In some embodiments, the organic solvent comprises isopropyl acetate or ethyl acetate.

In some embodiments, the solvent of (c) further comprises water in an amount that does not exceed the maximum solubility of water in the organic solvent. In some embodiments, the solvent of (c) further comprises water in an amount of less than about 3.5%. In some embodiments, an antisolvent is added to the filtered mixture of (c). In some embodiments, water is added to the filtered mixture of (c) in an amount of less than about 3.5%. In some embodiments, (c) is performed at or below about 20° C. to about 25° C., the filtered mixture from (c) is heated to ensure complete dissolution prior to the addition of water, and (d) is performed at or below about 20° C. to about 25° C. In some embodiments, (c) is performed at or below about 20° C. to about 25° C., the filtered mixture from (c) is heated to about 30° C.-50° C. to ensure complete dissolution prior to the addition of water, and (d) is performed at or below about 20° C. to about 25° C. In some embodiments, (c) is performed at about 20° C. to about 25° C., the filtered mixture from (c) is heated to about 45° C.-50° C. to ensure complete dissolution prior to the addition of water, and (d) is performed at about 20° C. to about 25° C. In some embodiments, (c) is performed at a temperature above about 20° C. to about 25° C. and (d) is performed at about 20° C. to about 25° C. or below about 20° C. to about 25° C. In some embodiments, (c) is performed at about 25-60° C. In some embodiments, (c) is performed at about 30° C.-50° C. In some embodiments, (c) is performed at about 45° C.-50° C. In some embodiments, (d) is performed at 0° C.-5° C.

Another aspect of the present disclosure provides a batch of pharmaceutical grade 2-Br-LSD, or a pharmaceutically acceptable salt thereof, prepared according to the methods of the disclosure. In some embodiments, the batch meets pre-set specifications that comprise a pre-set specification for iso-2-Br-LSD, wherein the pre-set specification for iso-2-Br-LSD is not more than 2%. In some embodiments, the batch meets pre-set specifications that comprise a pre-set specification for iso-2-Br-LSD, wherein the pre-set specification for iso-2-Br-LSD is not more than 0.4%. In some embodiments, the batch further comprises at least one of the following pre-set specifications: not more than 0.05% of LSD, not more than 0.5% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD. In some embodiments, the batch further comprises at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

The 2-Br-LSD may be isolated as a solid in accordance with standard techniques. In some embodiments, the isolating comprises collecting the solid via filtration. In some embodiments, the solid may be crystalline or amorphous, and if the former, have one or more polymorphic forms. In some embodiments, the crystalline solid may exist in one or more polymorphic forms. In some embodiments, the solid comprises crystalline solid 2-Br-LSD.

The isolating of solid 2-Br-LSD may include one or more purification steps, in accordance with standard techniques. In some embodiments, the one or more purification steps comprises chromatography. In some embodiments, the one or more purification steps comprises column chromatography. In some embodiments, the column chromatography is normal-phase. In some embodiments, the column chromatography is reversed-phase. In some embodiments, the one or more purification steps comprises crystallization. In some embodiments, the one or more purification steps comprises recrystallization. In some embodiments, the crystallization or recrystallization includes a filtration to remove impurities prior to crystallization. In some embodiments, the filtration is performed below about 20° C. to about 25° C. In some embodiments, the filtration is performed at about 20° C. to about 25° C. In some embodiments, the filtration is performed above about 20° C. to about 25° C. In some embodiments, the one or more purification steps comprises slurrying solid 2-Br-LSD in a solvent in which the impurities are more soluble than 2-Br-LSD, followed by separation of the solvent containing the impurities from the solid, purified 2-Br-LSD. In some embodiments, the crystallization, recrystallization or slurrying is performed on a salt of 2-Br-LSD. In some embodiments, the one or more purification steps comprises liquid-liquid extraction.

Another aspect of the present disclosure provides a batch of pharmaceutical grade 2-Br-LSD, or a pharmaceutically acceptable salt thereof, prepared according to the methods of the disclosure.

The 2-Br-LSD may be salified to produce a pharmaceutically acceptable salt by methods known in the art. The term “pharmaceutically acceptable salt” refers to a product obtained by reaction of the 2-Br-LSD with a suitable acid.

The purified solid 2-Br-LSD may also be solvated (i.e., with pharmaceutically acceptable solvents such as water, ethanol, and the like, for example, as a hydrate (e.g., mono-hydrate, di-hydrate, etc.)).

Another aspect of the present disclosure is directed to a pharmaceutical composition that comprises a therapeutically effective amount of pharmaceutical grade 2-Br-LSD, e.g., made by the disclosed process, or a pharmaceutically acceptable salt thereof, and a pharmaceutically acceptable carrier. In some embodiments, the 2-Br-LSD has a purity level of at least 98% area, as measured by HPLC/UV. In some embodiments, the 2-Br-LSD has a purity level of at least 99% area, as measured by HPLC/UV. The term “pharmaceutically acceptable carrier,” as known in the art, refers to a pharmaceutically acceptable material, composition, or vehicle, suitable for administering pharmaceutical grade 2-Br-LSD, or a pharmaceutically acceptable salt thereof, to mammals. Suitable carriers may include, for example, liquids (both aqueous and non-aqueous alike, and combinations thereof), solids, encapsulating materials, and combinations thereof (e.g., semi-solids). A carrier is “pharmaceutically acceptable” in the sense of being physiologically inert, compatible with the other ingredients of the formulation, and not injurious to the subject or patient. Depending on the type of formulation, the composition may further include one or more pharmaceutically acceptable excipients.

Broadly, pharmaceutical grade 2-Br-LSD and pharmaceutically acceptable salts thereof of the present disclosure may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

In some embodiments, 2-Br-LSD and pharmaceutically acceptable salts thereof are formulated for oral or intravenous administration (e.g., systemic intravenous injection).

In some embodiments, the 2-Br-LSD and pharmaceutically acceptable salts thereof may be formulated into solid compositions (e.g., powders, tablets, dispersible granules, capsules, cachets, and suppositories). In some embodiments, the 2-Br-LSD and pharmaceutically acceptable salts thereof may be formulated into liquid compositions (e.g., solutions in which the 2-Br-LSD and pharmaceutically acceptable salts thereof is dissolved, suspensions in which solid particles of the 2-Br-LSD and pharmaceutically acceptable salts thereof are dispersed, emulsions, and solutions containing liposomes, micelles, or nanoparticles, syrups and elixirs). The 2-Br-LSD and pharmaceutically acceptable salts thereof may also be formulated for rapid, intermediate or extended release.

Such compositions may be prepared in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

The 2-Br-LSD can be used for a variety of pharmacological uses. In some embodiments, the 2-Br-LSD may be used to treat disorders associated with pain, e.g., cephalic pain, vascular headaches, cluster headaches (see, e.g., U.S. Pat. No. 8,415,371, and to reduce stroke damage (see, e.g., U.S. Pat. No. 4,524,072).

These and other aspects of the present disclosure will be further appreciated upon consideration of the following Examples, which are intended to illustrate certain particular embodiments of the disclosure but are not intended to limit its scope, as defined by the claims.

### SPECIFIC EMBODIMENTS

Representative embodiments of the present disclosure are set forth in the following paragraphs.
Paragraph 1. A method of synthesizing pharmaceutical grade 2-bromolysergic acid diethylamide (2-Br-LSD), or a pharmaceutically acceptable salt thereof comprising:
   a. contacting lysergic acid with bromotrimethylsilane (TMSBr) and dimethylsulfoxide (DMSO), wherein the TMSBr and DMSO are in molar equivalents of about 6 and about 0.9, respectively, to form 2-bromolysergic acid;
   b. contacting the 2-bromolysergic acid with diethylamine and an amide coupling reagent, to form 2-Br-LSD;
   c. filtering a mixture of the 2-Br-LSD and a solvent;
   d. allowing the 2-Br-LSD to precipitate from the filtered mixture;
   e. collecting the 2-Br-LSD by filtration;
   f. analyzing the collected 2-Br-LSD of (e) for the presence of iso-2-Br-LSD; and
   g. (1) if a batch of the collected 2-Br-LSD of (e) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or
   g. (2) if a batch of the collected 2-Br-LSD of (e) fails to meet the pre-set specification for iso-2-Br-LSD, then purifying the collected 2-Br-LSD and repeating (f) and (g), or discarding the collected 2-Br-LSD.
Paragraph 2. The method of paragraph 1, wherein the pre-set specification for iso-2-Br-LSD is not more than 2%.
Paragraph 3. The method of paragraph 1 or 2, wherein the pre-set specification for iso-2-Br-LSD is not more than 0.4%.
Paragraph 4. The method of any one of paragraphs 1-3, further comprising at least one of the following pre-set specifications: not more than 0.05% of LSD, not more than 0.5% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.
Paragraph 5. The method of any one of paragraphs 1-4, further comprising at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.
Paragraph 6. The method of any one of paragraphs 1-5, wherein (a) comprises contacting the lysergic acid with TMSBr and DMSO in the presence of a solvent.
Paragraph 7. The method of paragraph 6, wherein the solvent is tetrahydrofuran (THF).
Paragraph 8. The method of any one of paragraphs 1-7, wherein (b) comprises contacting the 2-bromolysergic acid with diethylamine and an amide coupling reagent in the presence of a solvent.
Paragraph 9. The method of paragraph 8, wherein the solvent is THF.
Paragraph 10. The method of any one of paragraphs 1-9, wherein the amide coupling reagent of (b) is propylphosphonic anhydride (T3P), 2-(1H-benzotriazol-1-yl)-1,1,3,3-tetramethyluronium hexafluorophosphate (Hexafluorophosphate Benzotriazole Tetramethyl Uronium, or HBTU), 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium, or HATU), 1,1′-Carbonyldiimidazole (CDI), or acetic anhydride.
Paragraph 11. The method of any one of paragraphs 1-10, wherein the amide coupling reagent is T3P.
Paragraph 12. The method of any one of paragraphs 1-10, wherein the amide coupling reagent is HBTU.
Paragraph 13. The method of any one of paragraphs 1-12, wherein the solvent of (c) comprises an organic solvent.
Paragraph 14. The method of paragraph 13, wherein the organic solvent comprises isopropyl acetate or ethyl acetate.
Paragraph 15. The method of paragraph 13, wherein the solvent of (c) further comprises water in an amount of less than about 3.5%.
Paragraph 16. The method of paragraph 13, wherein water is added to the filtered mixture of (c) in an amount of less than about 3.5%.
Paragraph 17. The method of paragraph 16, wherein (c) is performed at or below about 20° C. to about 25° C., the filtered mixture from (c) is heated to ensure complete dissolution prior to the addition of water, and (e) is performed at or below about 20° C. to about 25° C.
Paragraph 18. The method of any one of paragraphs 1-16, wherein (c) is performed at a temperature above about 20° C. to about 25° C. and (d) is performed at or below about 20° C. to about 25° C.
Paragraph 19. The method of paragraph 18, wherein (c) is performed at about 30° C.-50° C.
Paragraph 20. The method of any one of paragraphs 1-19, wherein the purifying in (g2) comprises column chromatography.
Paragraph 21. The method of any one of paragraphs 1-20, wherein the purifying in (g2) comprises HPLC.
Paragraph 22. A batch of pharmaceutical grade 2-Br-LSD, or a pharmaceutically acceptable salt thereof, that meets pre-set specifications that comprise a pre-set specification for iso-2-Br-LSD, wherein the pre-set specification for iso-2-Br-LSD is not more than 0.4%.
Paragraph 23. The batch of paragraph 22, further comprising at least one of the following pre-set specifications: not more than 0.05% of LSD, not more than 0.5% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.
Paragraph 24. The batch of paragraph 22, further comprising at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.
Paragraph 25. A pharmaceutical composition, comprising the batch of any one of paragraphs 22-24, or a portion thereof, and a pharmaceutically acceptable carrier.

### EXAMPLES

### Example 1: HPLC Methods

### HPLC Method 1

System: Agilent 1100 series liquid chromatograph or equivalent
Column: Acquity BEH Phenyl 4.6×30 mm; 1.7 μm particle size
Mobile phase A: Water:Trifluoroacetic acid (100:0.03)
Mobile phase B: Acetonitrile:Trifluoroacetic acid (100:0.03)
Flow rate: 2.0 mL/min
Injection volume: 5 μL
Detection: 210 nm UV detection
Column temp.: 40° C.
Post run: 2.3 min

### Gradient:

| Time (mins) | % A | % B |
|---|---|---|
| 0 | 95 | 5 |
| 5 | 95 | 5 |
| 15 | 5 | 95 |
| 16 | 5 | 95 |
| 16.5 | 95 | 5 |
| 17 | 95 | 5 |

### Retention Times of Relevant Compounds:

| Compound | Retention time (min) | Relative retention time |
|---|---|---|
| HBTU | 0.8 | 0.10 |
| 2-Bromolysergic acid | 7.0 | 0.86 |
| HOBt | 7.2 | 0.89 |
| TMU | 7.4 | 0.91 |
| LSD | 7.8 | 0.96 |
| Iso-LSD | 8.0 | 0.99 |
| 2-Br-LSD | 8.1 | 1.00 |
| Iso-2-Br-LSD | 8.4 | 1.04 |

TMU = tetramethylurea

HPLC Method 2: Same as Method 1, but with detection at 254 nm.
HPLC Method 3: Same as Method 1, but with detection at 220 nm.

### HPLC Method 4:

System: Agilent 1100/1200 series liquid chromatograph or equivalent
Column: XSelect CSH C18; 150×4.6 mm, 3.5 μm particle size
Mobile phase A: Purified water:Acetonitrile:Trifluoroacetic acid (95:5:0.05%)
Mobile phase A: Acetonitrile:Purified water:Trifluoroacetic acid (95:5:0.05%)
Flow rate: 1.0 mL/min
Stop time: 21 min
Post run time: 5 min
Injection volume: 5 μL
Column temperature: 35° C.
Detection: UV 230 nm

### Gradient:

| Time | % A | % B |
|---|---|---|
| 0 | 100 | 0 |
| 3 | 100 | 0 |
| 15 | 20 | 80 |
| 20 | 20 | 80 |
| 21 | 100 | 0 |

### Retention Times of Relevant Compounds:

| | Retention | Relative | Concentration | |
|---|---|---|---|---|
| Compound | time (min) | retention time | of standard | Diluent |
| Lysergic acid | 8.27 | 0.80 | 0.30 mg/mL | methanol |
| 2-Bromolysergic | 9.11 | 0.88 | 0.30 mg/mL | methanol |
| acid | | | | |
| LSD | 9.85 | 0.95 | 0.30 mg/mL | methanol |
| Iso-LSD | 10.14 | 0.98 | 0.30 mg/mL | methanol |
| 2-Br-LSD | 10.37 | 1.00 | 0.30 mg/mL | methanol |
| Iso-2-Br-LSD | 10.83 | 1.04 | 0.30 mg/mL | methanol |

### Example 2: Synthesis of 2-Bromolysergic Acid Diethylamide Hemi-L-Tartrate

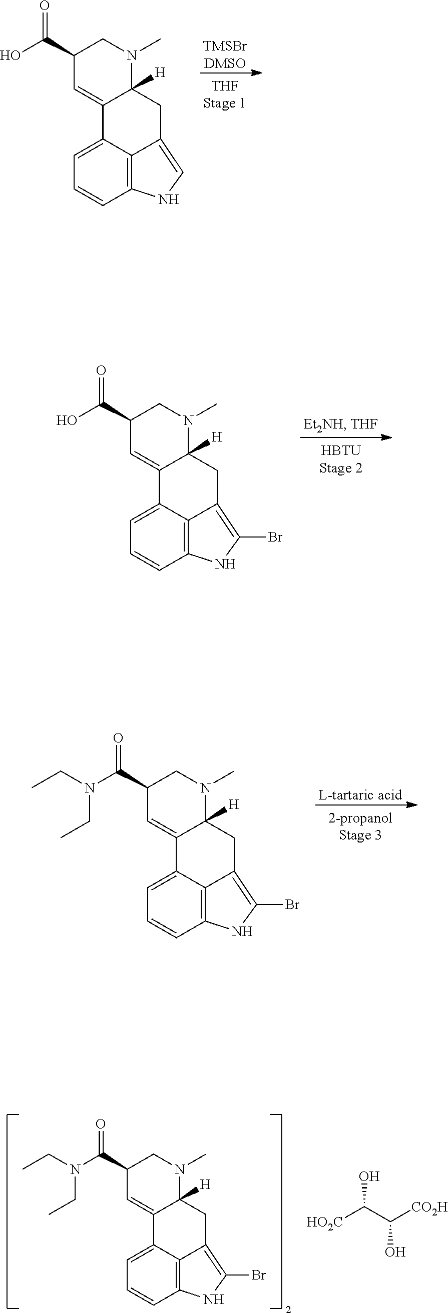

All processing steps were conducted under an atmosphere of nitrogen, unless indicated otherwise.

### Stage 1; Synthesis of 2-Bromolysergic Acid

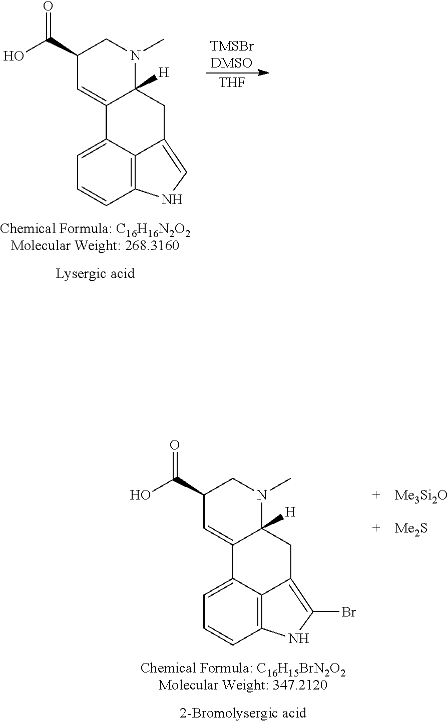

Lysergic acid (CAS RN 82-58-6, 300 g, 1.12 mol, limiting reagent) was suspended in tetrahydrofuran (6000 mL, 20 mL/g of lysergic acid). Stirring was commenced and the suspension was cooled to −25° C. to −10° C. (target: −15° C.). Anhydrous dimethyl sulfoxide (DMSO, 71.4 mL, 0.238 mL/g of lysergic acid, 1.01 mol, 0.9 equiv) was added. Bromotrimethylsilane (TMSBr, 97%, 868.2 mL, 2.894 mL/g of lysergic acid, 1027 g, 6.709 mol, 6.0 equiv) was added in one portion over about 30 see (target: <5 min, maximum temperature <10° C.), resulting in a ˜17° C. rise in the batch temperature from −13° C. to 4.4° C. (bath temperature −21)° C. The resulting grey suspension was stirred at 0° C.±10° C. for 25 to 35 min (target: 30 min; reaction time should not extend beyond 35 min including sampling for in-process analysis). The mixture was sampled for in-process analysis (HPLC Method 4, report results for 2-bromolysergic acid, lysergic acid) and then immediately diluted with heptane (3000 mL, 10 mL/g of lysergic acid) and filtered. Heptane (1200 mL, 4 mL/g of lysergic acid) was used to rinse the reaction vessel and wash the filtered solids. The solids were washed again with heptane (1200 mL, 4 mL/g of lysergic acid), pulled dry, and dried in a vacuum oven at 15° C. to 20° C. to yield a grey solid, 455.62 g; 1H NMR assay 72% (DMSO-d6 vs. 1,2,4,5-tetrachloro-3-nitrobenzene); active yield 328.0 g (84% yield); HPLC analysis (Method 4) 89.08% 2-bromolysergic acid, 7.53% lysergic acid; moisture by Karl Fischer titration 1.15%. Di-bromo- and tri-bromolysergic acids were undetectable by electrospray LCMS (HPLC Method 4).

### Stage 2: Synthesis of 2-Bromolysergic Acid Diethylamide (CAS RN 478-84-2)

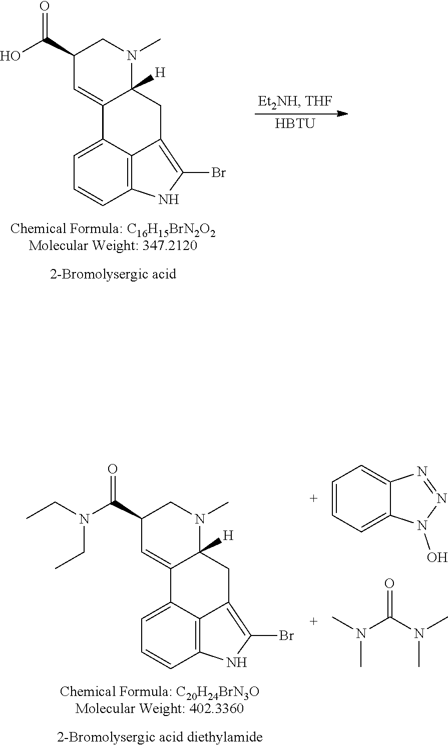

2-Bromolysergic acid (448.41 g, 1.29 mol, limiting reagent) was charged to a vessel, which was wrapped with aluminum foil to shield from light. Tetrahydrofuran (4.04 L, 9 mL/g of 2-bromolysergic acid) was charged and stirring was commenced to form a dark brown suspension. The batch temperature was adjusted to 15° C.±10° C. Diethylamine (587.9 mL, 1.311 mL/g of 2-bromolysergic acid, 5.68 mol, 4.4 equiv) was charged at a rate sufficient to maintain the batch temperature at 15±10° C. HBTU (646.6 g, 1.442 g/g of 2-bromolysergic acid, 1.70 mol, 1.3 equiv) was charged portion-wise over at least 10 minutes and at such a rate that the batch temperature was maintained at 15° C.±10° C. The batch was stirred for at least 1 hr at 15° C.±10° C. In-process analysis by HPLC Method 2 showed approximately 5% 2-bromolysergic acid remaining. Diethylamine (58.68 mL, 0.1309 mL/g of 2-bromolysergic acid, 0.5672 mol, 0.4 equiv) was charged to the vessel, followed by HBTU (53.12 g, 0.1185 g/g of 2-bromolysergic acid, 0.140 mol, 0.1 equiv), and stirring was continued for a further 15 minutes at 15° C.±10° C. HPLC analysis by Method 4 showed the following results:

| Compound | % Area |
|---|---|
| 2-Bromolysergic acid | 0.05% |
| LSD | 9.57% |
| Iso-LSD | 3.24% |
| 2-Br-LSD | 55.66% |
| Iso-2-Br-LSD | 13.97% |

The batch was filtered and ethyl acetate (˜500 mL) was used to rinse the vessel and wash the solids. The filtrate was concentrated in vacuo at 45° C. to dryness. The residue was charged back to the reaction vessel with ethyl acetate (5824 mL). The mixture was washed with pre-made K₂CO₃ (aq) solution (224 g in 2016 mL) by stirring for ˜5 minutes, allowing the mixture to stand, and separating the layers. The aqueous layer was charged back to the vessel and extracted in the same fashion with ethyl acetate (3.8 L). The combined organic layers were washed in the same fashion with pre-made K₂CO₃ (aq) solution (224 g in 2016 mL) followed by water (2.24 L). The organic layer was put into beakers and allowed to stand overnight, covered with foil and then concentrated in vacuo at 45° C., resulting in 648.55 g of residue, which was designated MK140C. HPLC analysis by Method 4 showed the following results:

HPLC Analysis of MK140C

| Compound | % Area |
|---|---|
| 2-Bromolysergic acid | Not detected |
| LSD | 10.38% |
| Iso-LSD | 4.01% |
| 2-Br-LSD | 60.31% |
| Iso-2-Br-LSD | 17.08% |

Isopropyl acetate (3.3 L, 7.5 mL/g of 2-bromolysergic acid) was charged to a clean vessel, followed by water (448 mL, 1 mL/g of 2-bromolysergic acid). The mixture was stirred for ˜5 minutes and allowed to stand until the layers separated. The lower aqueous layer was removed. The upper layer was set aside as “pre-wetted isopropyl acetate.” Residue MK140C was charged to the vessel, followed by pre-wetted isopropyl acetate (3.1 L, 7 mL/g of 2-bromolysergic acid), a portion of which was used to complete the transfer of MK140C. The stirrer was started, and the mixture was heated to 45° C. and stirred for ˜30 minutes. The mixture was allowed to cool to 0° C. over 2 hours and stirred for ˜1 hour. The precipitated solid was filtered, washed with three ˜500-mL portions of cold (0° C.-5° C.)isopropyl acetate, and designated MK140D. HPLC analysis by Method 4 showed the following results:

HPLC Analysis of MK140D

| Compound | % Area |
|---|---|
| LSD | 9.77% |
| Iso-LSD | 0.86% |
| 2-Br-LSD | 71.99% |
| Iso-2-Br-LSD | 16.65% |

The filtrate was concentrated and the residue, designated MK140D-FL, analyzed by HPLC Method 4 to give the following results:

HPLC Analysis of MK140D-FL

| Compound | % Area |
|---|---|
| LSD | 12.23% |
| Iso-LSD | 11.76% |
| 2-Br-LSD | 27.15% |
| Iso-2-Br-LSD | 24.49% |

Chromatography on silica gel, with 0.1% diethylamine in ethyl acetate as eluent, purged LSD and iso-LSD from 2-Br-LSD; however, significant amounts of iso-2-Br LSD remained (data not shown). Therefore, lot MK140D, containing iso-2-Br-LSD at 16.65% area, would be unsuitable for progression to pure 2-Br-LSD unless a method could be found to purge iso-2-Br-LSD. In a previous experiment (data not shown), recrystallization of crude 2-Br-LSD, prepared as described above, from wet isopropyl acetate, resulted in purging of iso-2-Br-LSD to a level of only 0.37%; however, experiment MK 140D and others described below showed that this result was not reliably reproducible. The following experiments illustrate that the inclusion of a filtration step (as opposed to no filtration) results in a reproducible method for achieving this level of iso-2-Br-LSD.

A flask was charged with lot MK140D (10 g) and pre-wetted isopropyl acetate (85 mL, 8.5 mL/g of crude 2-Br-LSD). The mixture was heated with stirring to 45° C. and maintained at this temperature for about 30 minutes. The solution was cooled slowly to 0° C. and then stirred for approximately 45 minutes. The mixture was filtered and the solid washed with cold (0° C.-5° C.) isopropyl acetate, pulled dry on the filter, designated MK141A, and analyzed by HPLC Method 3 to give the following results:

HPLC Analysis of MK141A

| Compound | % Area |
|---|---|
| LSD | 9.10% |
| Iso-LSD | Not detected |
| 2-Br-LSD | 76.59% |
| Iso-2-Br-LSD | 14.24% |

The HPLC results for MK141A indicated that recrystallization from pre-wetted isopropyl acetate did not result in purging iso-2-Br-LSD from MK140D. Sample MK141A, the corresponding filtrate, the remainder of MK140D, and MK140D-FL were combined and concentrated in vacuo to afford 590 g of residue, which was designated MK140D-R.

A flask was charged with MK140D-R (10 g) and pre-wetted isopropyl acetate (85 mL, 8.5 mL/g of crude 2-Br-LSD). The mixture was heated with stirring to 45° C. and maintained at this temperature for about 30 minutes. The solution was cooled slowly over 2 hours to 0° C. and then stirred for approximately 45 minutes. The mixture was filtered and the solid washed with cold (0° C.-5° C.)isopropyl acetate (3×10 mL), pulled dry on the filter, designated MK142-1A, and analyzed by HPLC Method 3 to give the following results:

HPLC Analysis of MK142-1A

| Compound | % Area |
|---|---|
| LSD | 9.88% |
| Iso-LSD | 0.37% |
| 2-Br-LSD | 76.32% |
| Iso-2-Br-LSD | 12.18% |

A flask was charged with MK140D-R (10 g) and pre-wetted ethyl acetate (prepared from ethyl acetate and water as described above for pre-wetted isopropyl acetate, 85 mL, 8.5 mL/g of crude 2-Br-LSD). The mixture was heated with stirring to 45° C. and maintained at this temperature for about 30 minutes. The solution was cooled slowly over 2 hours to 0° C. and then stirred for approximately 45 minutes. The mixture was filtered and the solid washed with cold (0° C.-5° C.)ethyl acetate (3×10 mL), pulled dry on the filter, designated MK142-2A, and analyzed by HPLC Method 3 to give the following results:

HPLC Analysis of MK142-2A

| Compound | % Area |
|---|---|
| LSD | 12.46% |
| Iso-LSD | 0.21% |
| 2-Br-LSD | 76.41% |
| Iso-2-Br-LSD | 10.68% |

The HPLC results for MK142-1A and MK142-2A indicated that recrystallization from pre-wetted isopropyl acetate or ethyl acetate, respectively, did not result in purging iso-2-Br-LSD from MK140D-R.

Two recrystallization experiments (JCCA2770-1 and JCCA2770-2) were set up in parallel, as follows. Pre-wetted isopropyl acetate was prepared from isopropyl acetate (120 mL) and water (20 mL) as described above. Two flasks were each charged with MK140D-R (10 g) and pre-wetted isopropyl acetate (53 mL, 5.3 mL/g of crude 2-Br-LSD). Each mixture was heated to about 50° C. The mixture in flask 1 was filtered while warm through Whatman GF/F filter paper (0.7 μm pore size), and the filtrate was re-heated to 50° C. The mixture in flask 2 was not filtered. Both mixtures were cooled slowly while stirring and sampled upon reaching 20° C., 10° C., 0° C.-5° C. and after 1 hour at 0° C.-5° C. Each sample was filtered and the collected solids analyzed by HPLC Method 3 to give the following results:

HPLC Analysis of JCCA2770-1 and JCCA2770-2
Process: Recrystallization with hot filtration (JCCA2770-1)

| | Sample Code: | | | |
|---|---|---|---|---|
| | JCCA2770- | JCCA2770- | JCCA2770- | JCCA2770- |
| | 1A | 1B | 1C | 1D |
| | Sampling temperature: | | | |
| | | | | After 1 hour |
| | 20° C. | 10° C. | 0-5° C. | at 0-5° C. |
| Compound | % Area | % Area | % Area | % Area |
| LSD | 8.12% | 8.06% | 8.89% | 9.30% |
| Iso-LSD | ND | ND | 0.47% | ND |
| 2-Br-LSD | 89.29% | 90.15% | 87.52% | 88.74% |
| Iso-2-Br-LSD | 2.04% | 1.42% | 2.46% | 1.46% |

Process: Recrystallization with hot filtration (JCCA2770-2)

| | JCCA2770- | JCCA2770- | JCCA2770- | JCCA2770- |
|---|---|---|---|---|
| Sample Code: | 2A | 2B | 2C | 2D |
| Sampling | | | | After 1 hour |
| temperature: | 20° C. | 10° C. | 0-5° C. | at 0-5° C. |
| Compound | % Area | % Area | % Area | % Area |
| LSD | 6.15% | 6.46% | 7.91% | 8.01% |
| Iso-LSD | 0.74% | 0.55% | 0.49% | ND |
| 2-Br-LSD | 74.31% | 74.63% | 74.45% | 74.29% |
| Iso-2-Br-LSD | 18.65% | 18.07% | 16.91% | 17.34% |

ND = not detected

The HPLC results for JCCA2770-1A through-1D indicated that filtration of a warm suspension of MK140D-R in pre-wetted isopropyl acetate, followed by recrystallization, was effective in purging iso-2-Br-LSD. The same procedure without the filtration (JCCA2770-2A through-2D) did not result in purging iso-2-Br-LSD.

Lots MK142-1A and MK142-2A were combined with the remainder of MK140D-R, and the solvent removed in vacuo, to afford 570 g of residue. Wet isopropyl acetate (3033 mL) was charged, and the mixture was heated with stirring to 45° C.-50° C. The warm mixture was filtered. The filtered solids (designated MK140E), were analyzed by HPLC Method 4, with the following results:

HPLC Analysis of MK140E

| Compound | % Area |
|---|---|
| LSD | 1.15% |
| Iso-LSD | 4.50% |
| 2-Br-LSD | 7.22% |
| Iso-2-Br-LSD | 86.43% |

The filtrate was charged back to the vessel, equilibrated at 45° C.±5° C., cooled to 0° C.-5° C. over 2 hours, stirred at 0° C.-5° C. for 1 hour and filtered. The solid product was washed with cold (0° C.-5° C.) isopropyl acetate (3×448 mL) and pulled dry to afford a brown solid, 226.11 g, which was designated MK140F. Analysis of MK140F by HPLC Method 4 gave the following results:

HPLC Analysis of MK140F

| Compound | % Area |
|---|---|
| LSD | 10.65% |
| Iso-LSD | 0.03% |
| 2-Br-LSD | 87.83% |
| Iso-2-Br-LSD | 0.23% |

The HPLC result for MK140F indicated that filtration of a warm suspension of MK140D-R in pre-wetted isopropyl acetate, followed by recrystallization, was effective in purging iso-2-Br-LSD.

Lot MK140F (226 g) was charged to a clean rotary evaporator flask with ethyl acetate (1130 mL, 5 mL/g of MK140F) and silica gel (452 g, 2 g/g of MK140F). This mixture was concentrated in vacuo at 40-50° C. to dryness. Fresh silica gel (1357 g, 6 g/g of MK140F) slurried in ethyl acetate was packed in a column and the sample, adsorbed on silica gel, was layered on top. The column was eluted with 0.1% v/v diethylamine in ethyl acetate and twelve 2-L fractions were collected. Fractions 3 to 11, containing 2-Br-LSD, were combined and concentration in vacuo at about 50° C. to afford 158.4 g (30.5% yield) of off-white foamy solid, which was designated MK140G. Analysis of MK140G by HPLC Method 4 gave the following results:

HPLC Analysis of MK140G

| Compound | % Area |
|---|---|
| LSD | 0.02% |
| Iso-LSD | ND |
| 2-Br-LSD | 99.63% |
| Iso-2-Br-LSD | 0.05% |

The HPLC result for MK140G indicated that chromatography was effective in purging LSD and iso-LSD. Additionally, although chromatography was not as effective in purging large amounts of iso-2-Br-LSD from 2-Br-LSD, chromatography was effective in reducing smaller amounts of iso-2-Br-LSD, for example, from 0.23% to 0.05%.

### Stage 3: Synthesis of 2-Bromolysergic Acid Diethylamide Hemi-L-Tartrate (CAS 4043-81-6)

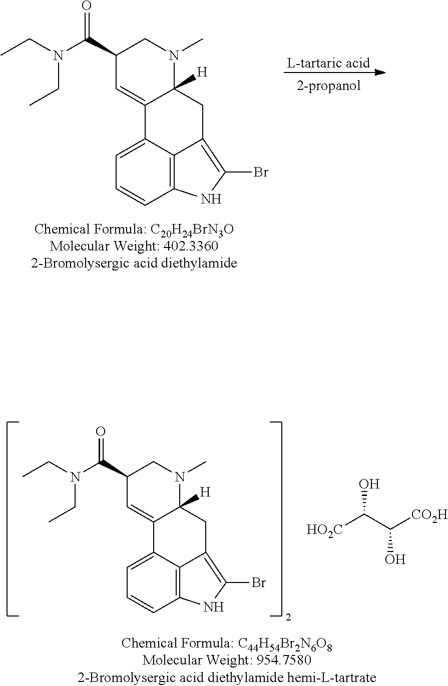

2-Bromolysergic acid diethylamide (Lot MK140G, 98.2 g, limiting reagent) was charged to a vessel and 2-propanol (1365 mL, 13.9 mL/g of 2-Br-LSD) was added, resulting in a brown solution. A solution of L-tartaric acid (18.4 g, 0.1875 g/g of 2-Br-LSD, 0.5 equiv) in 2-propanol (196 mL, 2 mL/g of 2-Br-LSD) was added at 20° C. over 20 minutes, initially producing thick yellow solids, which converted to mobile off-white solids. The mixture was stirred for an additional 30 minutes at ambient temperature (e.g., about 20° C. to about 25° C.), and then filtered. The solids were washed with 2-propanol (2×196 mL, 2×2 mL/g of 2-Br-LSD) and dried in vacuo at 70° C. to give 102.29 g (87.8% yield; 31.4% from lysergic acid) of white solid, which was designated MK140H. Analysis by HPLC Method 4 showed 99.93% chemical purity with 0.02% LSD, 0.02% iso-2-Br-LSD, and 0.07% total impurities. FTIR: 1627 cm-1 (amide carbonyl) and 1444 cm-1 (carboxylic acid carbonyl). Electrospray MS: m/z 402.3, 404.3 ([MH]⁺). L-Tartaric acid content by 1H NMR: 16.37%. PF₆ content by 19F NMR: <1500 ppm. Residual solvents by headspace GC: 2-propanol 1197 ppm; ethyl acetate 1 ppm; THF<7 ppm; isopropyl acetate<4 ppm; heptane<67 ppm. TMU by HPLC<750 ppm. Residue on ignition: not detected. Elemental impurities by ICP-MS: Cu 1.1 ppm; Ni 0.9 ppm; As, Cd, Co, Hg, Li, Pb, Sb, V each <0.1 ppm. 1H and ¹³C NMR spectra of MK140H were obtained using a Jeol ECX400 spectrometer. Chemical shifts are reported in ppm relative to solvent used (DMSO-d₆: δ=2.50 ppm (¹H NMR), δ=39.51 ppm (¹³C NMR)). Analysis of MK140H by ¹H and ¹³C NMR gave data that are consistent with the proposed structure. The ¹H and ¹³C NMR spectra were assigned and showed all the expected characteristic signals (see Tables 1 and 2).

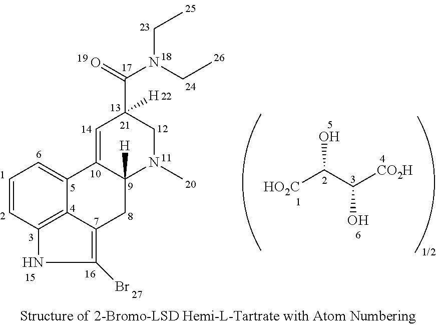

TABLE 1
¹H NMR Table of Assignments for MK140H

| | | | Coupling | Peak Assignment |
|---|---|---|---|---|
| | Number | | Constants/ | (atom number as per |
| δ/ppm | of H | Multiplicity | Hz | structure above) |
| 1.05 | 3 | t | 7.0 | 2 × CH₃ (25, 26) |
| 1.17 | 3 | t | 7.0 | |
| 2.44 | 1 | dd | 14.7, 11.0 | 1 × CHH (8) |
| 2.53 | 3 | s | — | 1 × CH₃ (20) |
| 2.69 | 1 | t | 10.8 | 1 × CHH (12) |
| 3.05 | 1 | dd | 11.0, 5.0 | 1 × CHH (12) |
| 3.12-3.21 | 1 | m | — | 1 × CH (21) |
| 3.24-3.37 | 3 | m | — | 2 × CH₂ (23, 24) and |
| 3.44 | 2 | q | 7.0 | 1 × CHH (8) |
| 3.80-3.87 | 1 | m | | CH (22) |
| 4.23 | 1 | s | — | Tartaric acid |
| | | | | 2 × CH (2, 3) |
| | | | | Hemi Salt |
| 6.28 | 1 | s | — | 1 × CH (14) |
| 7.05-7.17 | 2 | m | — | 3 × CH (1, 6, 2) |
| 11.51 | 1 | s | | 1 × NH (15) |

TABLE 2
¹³C Table of Assignments for MK140H

| | Peak Assignment (atom |
|---|---|
| δ/ppm | number as per structure above) |
| 13.08 | 2 × CH₃ (25, 26) |
| 14.83 | |
| 25.85 | CH₂ (8) |
| 36.69 | CH (13) |
| 39.57, | 2 × CH₂ (23, 24) |
| 41.58 | |
| 43.09 | CH₃ (20) |
| 55.38 | CH₂(12) |
| 61.97 | CH (9) |
| 72.07 | Tartaric acid 2 × CH (2, 3) |
| 103.87 | C (7) |
| 108.97 | C (16) |
| 109.35, | 3 × CH (1, 2, 6) |
| 112.10, | |
| 122.90 | |
| 120.86 | CH (14) |
| 125.71 | C (4) |
| 126.15 | C (5) |
| 133.85 | C (10) |
| 134.22 | C (3) |
| 170.33 | CON (17) |
| 173.53 | Tartaric acid 2 × CO₂H (1, 4) |

### Solid State Assignment of MK140H:

X-Ray Powder Diffraction (XRPD) patterns were collected on a PANalytical diffractometer using Cu Kα radiation (45 kV, 40 mA). The data collection range was 2.994-35° 2θ with a continuous scan speed of 0.2° s⁻¹.

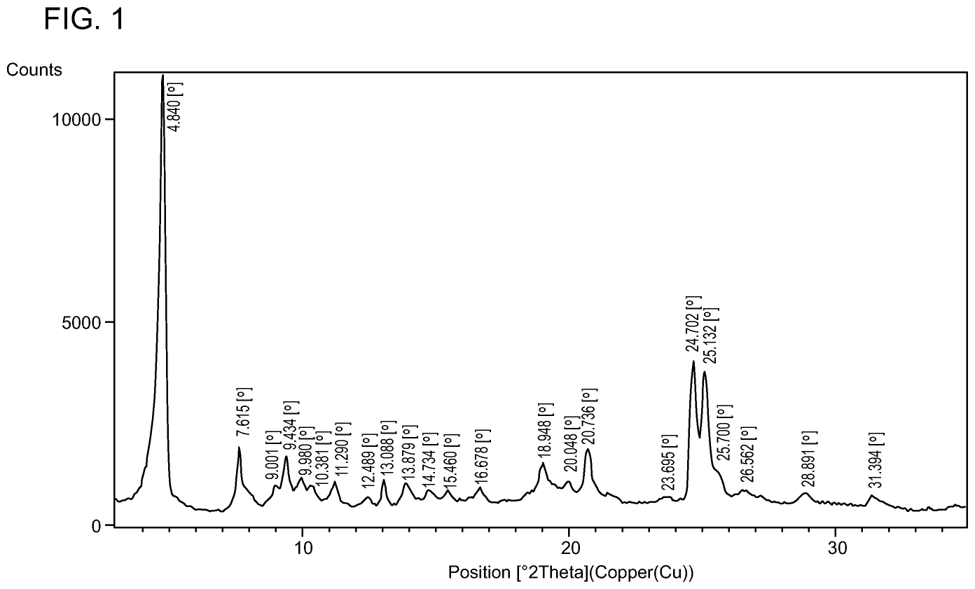

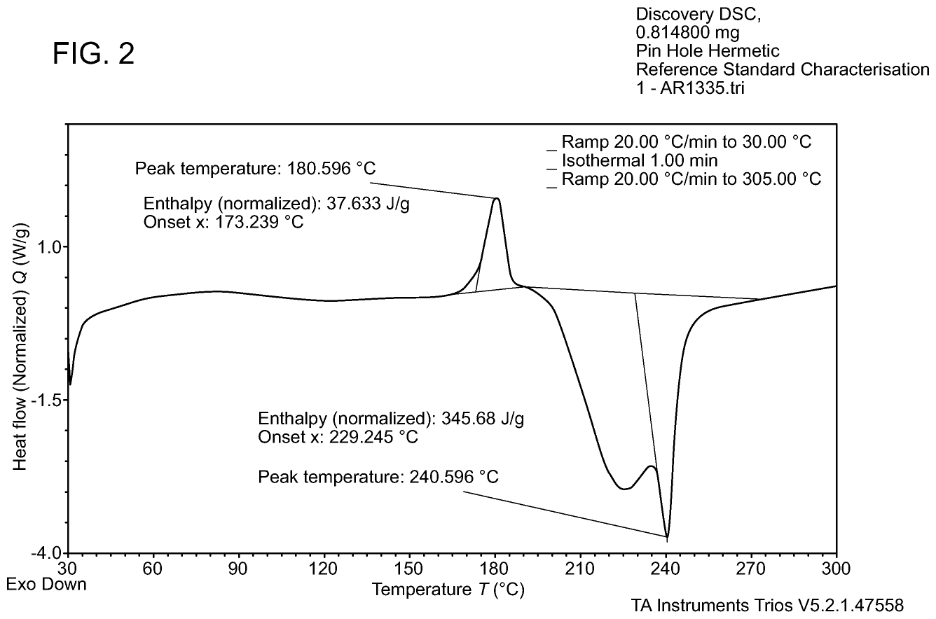

Differential scanning calorimetry (DSC) data was collected on a TA Discovery DSC and heated at 20° C./min from 30 to 305° C. The DSC thermograph of MK140H showed a main endotherm at 180.6° C. with an onset at 173.2° C. The XRP diffractogram and DSC thermograph of MK140H indicated a crystalline solid consistent with the preferred form identified during development (FIG. 1 and FIG. 2).

### Example 3: Synthesis of 2-Bromolysergic Acid Diethylamide Hemi-L-Tartrate

The following process was performed in accordance with GMP appropriate for early-stage clinical use as defined in Q7. All processing steps were conducted under an atmosphere of nitrogen, unless indicated otherwise.

### Stage 1: Synthesis of 2-Bromolysergic Acid

Lysergic acid (CAS RN 82-58-6, 299.9 g, 1.12 mol, limiting reagent) was suspended in tetrahydrofuran (5998 mL, 20 mL/g of lysergic acid). Stirring was commenced and the suspension was cooled to −25° C. to −10° C. (target: −15° C.). Anhydrous dimethyl sulfoxide (DMSO, 71 mL, 0.238 mL/g of lysergic acid, 1.00 mol, 0.9 equiv) was added. Bromotrimethylsilane (TMSBr, 97%, 868 mL, 2.894 mL/g of lysergic acid, 1,027 g, 6.71 mol, 6.0 equiv) was added in one portion over about 30 sec (target: <5 min, maximum temperature <10° C.), resulting in a ˜10.2° C. rise in the batch temperature from −13.8° C. to −3.6° C. The resulting grey suspension was stirred at 0° C.±10° C. for 25 to 35 min (target: 30 min; reaction time should not extend beyond 35 min including sampling for in-process analysis). The mixture was sampled for in-process analysis (HPLC Method 4, report results for 2-bromolysergic acid, lysergic acid) and then immediately diluted with heptane (2999 mL, 10 mL/g of lysergic acid) and filtered. Heptane (1200 mL, 4 mL/g of lysergic acid) was used to rinse the reaction vessel and wash the filtered solids. The solids were washed again with heptane (1200 mL, 4 mL/g of lysergic acid), pulled dry, and dried in a vacuum oven at 15° C. to 20° C. to yield a grey solid, 452.6 g; 1H NMR assay 65% (DMSO-d₆ vs. 1,2,4,5-tetrachloro-3-nitrobenzene); active yield 294.2 g (76% yield); moisture by Karl Fischer titration 1.21%. Analysis by HPLC Method 4 showed the following results:

| Compound | Specification | Result |
|---|---|---|
| Lysergic acid | Report result—typically | 14.67% area |
| | NMT 10% area | |
| 2-Bromolysergic | Report result—typically | 80.29% area |
| acid | NLT 88.0% area | |

NMT = not more than;
NLT = not less than

Although the lysergic acid content was higher than expected based on development results, the batch was progressed to the next stage. Lysergic acid was expected to convert to LSD in Stage 2, which could be removed by chromatographic purification of 2-Br-LSD.

### Stage 2: Synthesis of 2-Bromolysergic Acid Diethylamide

2-Bromolysergic acid (444.3 g, 1.28 mol, limiting reagent) was charged to a vessel, which was shielded from light. Tetrahydrofuran (3999 mL, 9 mL/g of 2-bromolysergic acid) was charged and stirring was commenced to form a suspension. The batch temperature was adjusted to 15±10° C. Diethylamine (540 mL, 1.22 mL/g of 2-bromolysergic acid, 5.22 mol, 4.1 equiv) was charged at a rate sufficient to maintain the batch temperature at 15° C.±10° C. HBTU (590.9 g, 1.330 g/g of 2-bromolysergic acid, 1.56 mol, 1.2 equiv) was charged portion-wise over at least 10 minutes and at such a rate that the batch temperature was maintained at 15° C.±10° C. Tetrahydrofuran (450 mL) was used to rinse any residual HBTU into the reaction flask at 15° C.±10° C. The batch was stirred for 63 minutes, during which time the temperature reached a maximum of 29.5° C. In-process analysis by HPLC Method 1 showed the following results:

| Compound | Specification | Result |
|---|---|---|
| 2-Bromolysergic | NMT 1.5% area | 0.19% area |
| acid | | |
| LSD | Report result—typically | 15.86% area |
| | NMT 10% area | |
| Iso-LSD | Report result—typically | 5.13% area |
| | NMT 3% area | |
| 2-Br-LSD | Report result—typically | 61.34% area |
| | NLT 60.0% area | |
| Iso-2-Br-LSD | Report result—typically | 14.41% area |
| | NMT 20% area | |

NMT = not more than;
NLT = not less than

The batch was filtered and ethyl acetate (2×900 mL) was used to rinse the vessel and wash the solids. The filtrate was transferred to a rotary evaporator flask, using ethyl acetate (500 mL) to complete the transfer, and concentrated in vacuo at 50° C. to dryness, giving a dark oil. The reaction vessel was cleaned with ethyl acetate, and the batch residue was charged back into it, using ethyl acetate (5×1000 mL and 1×700 mL) to aid the transfer, warming as necessary to 40° C.-50° C. to mobilize the residue. The batch was heated to 30° C.-50° C. (target 40° C.) to give a solution, and maintained at this temperature during the following extraction steps. The batch was washed with pre-made K₂CO₃ (aq) solution (222.8 g in 2000 mL) by stirring for ˜5 minutes, allowing the mixture to stand, and separating the layers. The aqueous layer was charged back to the vessel and extracted in the same fashion with ethyl acetate (2750 mL). The combined organic layers were washed in the same fashion with pre-made K₂CO₃ (aq) solution (212.9 g in 2000 mL) followed by water (3200 mL). The organic layer was transferred to a rotary evaporator flask, using ethyl acetate (3×200 mL) to complete the transfer, and concentrated in vacuo at 45° C., resulting in an oil.

Isopropyl acetate (3390 mL, 7.6 mL/g of 2-bromolysergic acid) was charged to a clean vessel, followed by water (450 mL, 1 mL/g of 2-bromolysergic acid). The mixture was stirred for ˜5 minutes and allowed to stand until the layers separated. The lower aqueous layer was removed. The upper layer was set aside as “pre-wetted isopropyl acetate.” The batch was charged to the vessel, using pre-wetted isopropyl acetate (3170 mL in 4 portions, 7 mL/g of 2-bromolysergic acid) and warming to 40° C.-50° C. as necessary to complete the transfer. The stirrer was started, and the mixture was heated to 46.1° C. (target range 40-50° C.) and stirred for ˜20 minutes until no further dissolution was observed. The mixture was filtered through GF/F filter paper in a Büchner funnel while maintaining the batch temperature above 35° C. The filtrate was transferred to a clean flask and heated to 40° C.-50° C. with stirring to ensure that all solids dissolved (final temperature 43.3° C.). The mixture was allowed to cool to 4.6° C. (target range 0° C.-5° C.) over 128 minutes (target at least 2 hours) and stirred at 0° C.-5° C. for 81 minutes (final temperature 2.8° C.; target stir time at least 1 hour; maximum stir time 1.5 hour). The precipitated solid was filtered, washed with cold (0° C.-5° C.)isopropyl acetate, 3×450 mL (1 mL/g of 2-bromolysergic acid), and pulled dry on the filter for at least 30 minutes. In-process HPLC analysis of the solid (designated Step 48 filter cake) by Method 4 showed the following results:

| Compound | Specification | Result |
|---|---|---|
| 2-Bromolysergic | Report result—typically | ND |
| acid | ND | |
| LSD | Report result—typically | 18.68% area |
| | NMT 10% area | |
| Iso-LSD | Report result—typically | 0.45% area |
| | ND | |
| 2-Br-LSD | Report result—typically | 74.20% area |
| | NLT 88% area | |
| Iso-2-Br-LSD | NMT 0.4% area | 5.50% area |

NMT = not more than;
NLT = not less than;
ND = not detected

The result for iso-2-Br-LSD (5.50% area) failed the in-process specification of NMT 0.4% area. In order to identify a method to further reduce the level of iso-2-Br-LSD, two non-GMP use tests were performed. In one use test, a sample of the Step 48 filter cake was subjected to the same recrystallization conditions with hot filtration as described in the previous paragraph, with all quantities adjusted proportionally. Analysis of the resulting filter cake by HPLC Method 4 showed no change in the purity of the material. In the second use test, the samples of the Step 48 filter cake and the corresponding filtrate (concentrated) were recombined in the same ratio to their proportion in the bulk material. This blend was recrystallized with hot filtration as described in the previous paragraph, with all quantities adjusted proportionally. Analysis of the resulting filter cake by HPLC Method 4 showed 71.57% 2-Br-LSD, 22.33% LSD and 1.82% iso-2-Br-LSD. Although the level of iso-2-Br-LSD still did not meet the target of NMT 0.4%, it was reduced from 5.50% to 1.82%. This filter cake was subjected to a further use test of the chromatographic purification on silica gel, eluting with 0.1% diethylamine in ethyl acetate, that normally follows the recrystallization in the GMP process (see below). Fractions 3 to 8 were combined, concentrated, and analyzed by HPLC Method 4, which showed 97.8% 2-Br-LSD, 0.16% LSD and 1.4% iso-2-Br-LSD. On the basis of these use test results, it was decided to implement Temporary Processing Instructions to blend the bulk Step 48 filter cake with the corresponding concentrated filtrate and repeat the recrystallization with hot filtration before proceeding to the chromatographic purification as instructed in the Batch Production Record.

Isopropyl acetate (3570 mL, 7.5 mL/g of 2-Br-LSD [sum of filter cake and concentrated filtrate from Step 48=476.0 g]) was charged to a clean vessel, followed by water (480 mL, 1 mL/g of 2-Br-LSD). The mixture was stirred for ˜5 minutes and allowed to stand until the layers separated. The lower aqueous layer was removed. The upper layer was set aside as “pre-wetted isopropyl acetate.” The Step 48 filter cake and concentrated filtrate were charged to the vessel, using pre-wetted isopropyl acetate (3330 mL in 4 portions, 7 mL/g of 2-bromolysergic acid) and warming to 40° C.-50° C. as necessary to complete the transfer. The stirrer was started, and the mixture was heated to 47.0° C. (target range 40° C.-50° C.) and stirred for ˜25 minutes until no further dissolution was observed. The mixture was filtered through GF/F filter paper in a Büchner funnel while maintaining the batch temperature above 35° C. The filtrate was transferred to a clean flask and heated to 40° C.-50° C. with stirring to ensure that all solids dissolved (final temperature 49.6° C.). The mixture was allowed to cool to 5.0° C. (target range 0° C.-5° C.) over 135 minutes (target at least 2 hours) and stirred at 0° C.-5° C. for 60 minutes (final temperature 1.9° C.; target stir time at least 1 hour; maximum stir time 1.5 hour). The precipitated solid was filtered, washed with cold (0° C.-5° C.)isopropyl acetate, 3×480 mL (1 mL/g of 2-Br-LSD), and pulled dry on the filter for at least 30 minutes. In-process HPLC analysis of the filter cake by Method 4 showed the following results:

| Compound | Specification | Result |
|---|---|---|
| 2-Bromolysergic | Report result—typically | ND |
| acid | ND | |
| LSD | Report result—typically | 22.47% area |
| | NMT 10% area | |
| Iso-LSD | Report result—typically | 0.61% area |
| | ND | |
| 2-Br-LSD | Report result—typically | 75.20% area |
| | NLT 88% area | |
| Iso-2-Br-LSD | NMT 0.4% area | 0.51% area |

NMT = not more than;
NLT = not less than;
ND = not detected

Although the in-process result for iso-2-Br-LSD (0.51% area) slightly exceeded the specification of NMT 0.4% area, it was decided to proceed with the subsequent steps of the Batch Production Record.

The recrystallized 2-Br-LSD (167.5 g, mass used to calculate charges for chromatographic purification) was charged to a rotary evaporator flask, followed by silica (336.9 g, 2 g/g of 2-Br-LSD) and ethyl acetate (840 mL, 5 mL/g of 2-Br-LSD). The flask was warmed at 50° C. on the rotary evaporator to dissolve the crude product, and then vacuum was carefully applied and the material concentrated to dryness. A sintered funnel was loaded with silica (1005.4 g, 6 g/g of 2-Br-LSD) slurried in ethyl acetate (3×1000 mL) while ensuring that the silica was packed tightly with no cracks. The silica having the crude 2-Br-LSD adsorbed was applied evenly to the top of the silica pad in the funnel. The product was eluted with 0.1% diethylamine in ethyl acetate (21978 mL in 11 portions), while collecting fractions of approximately 1675 mL (10 mL/g of 2-Br-LSD). The fractions were analyzed by thin layer chromatography (TLC) on silica with 0.1% diethylamine in ethyl acetate as eluent, using UV light at 254 nm for visualization (2-Br-LSD dark blue spot Rf˜0.3; LSD bright blue spot Rf˜0.1) and, as needed, HPLC Method 4. Fractions 2-6 containing 2-Br-LSD were combined, transferred via a Supapore capsule PP 1.2 μm in-line filter into a rotary evaporator, rinsing with ethyl acetate (4×100 mL and 2×200 mL), and the solvent was removed in vacuo at a bath temperature of 50° C. to constant weight (<0.5% change upon an additional 30 minutes of rotary evaporation in vacuo) to give 100.6 g of yellow solid. Analysis by HPLC Method 4 showed the following results:

| Compound | Specification | Result |
|---|---|---|
| 2-Bromolysergic | Report result—typically | ND |
| acid | ND | |
| LSD | NMT 0.05% area | 0.10% area |
| Iso-LSD | Report result—typically | ND |
| | ND | |
| 2-Br-LSD | Report result—typically | 98.95% area |
| | NLT 99.0% area | |
| Iso-2-Br-LSD | Report result—typically | 0.03% area |
| | NMT 0.1% area | |

NMT = not more than;
NLT = not less than;
ND = not detected

Although chromatographic purification successfully reduced the level of iso-2-Br-LSD to 0.03%, the result for LSD (0.10% area) failed the specification of NMT 0.05% area for release of the 2-Br-LSD intermediate. Therefore, it was decided to implement Temporary Processing Instructions to repeat the chromatographic purification, in order to reduce the LSD content to within the specification limit.

The previously chromatographed 2-Br-LSD (98.6 g, mass used to calculate charges for chromatographic purification) was charged to a rotary evaporator flask, followed by silica (198.0 g, 2 g/g of 2-Br-LSD) and ethyl acetate (500 mL, 5 mL/g of 2-Br-LSD). The flask was warmed at 50° C. on the rotary evaporator to dissolve the crude product, and then vacuum was carefully applied and the material concentrated to dryness. A sintered funnel was loaded with silica (788.7 g, 8 g/g of 2-Br-LSD) slurried in ethyl acetate (1000 mL plus 2×500 mL) while ensuring that the silica is packed tightly with no cracks. The silica having 2-Br-LSD adsorbed was applied evenly to the top of the silica pad in the funnel. The product was eluted with 0.1% diethylamine in ethyl acetate (13986 mL in 7 portions), while collecting fractions of approximately 493 mL (5 mL/g of 2-Br-LSD). Analysis of the fractions by TLC as described above was used to select fractions for analysis by HPLC Method 4. Fractions 6-22 containing 2-Br-LSD were combined, transferred via a Supapore capsule PP 1.2 μm in-line filter into a rotary evaporator, rinsing with ethyl acetate (250 mL), and the solvent was removed in vacuo at a bath temperature of 50° C. to give 88.2 g (17.1% yield) of yellow solid. Analysis by HPLC Method 4 showed the following results:

| Compound | Specification | Result |
|---|---|---|
| 2-Bromolysergic | Report result—typically | ND |
| acid | ND | |
| LSD | NMT 0.05% area | ND |
| Iso-LSD | Report result—typically | ND |
| | ND | |
| 2-Br-LSD | Report result—typically | 99.17% area |
| | NLT 99.0% area | |
| Iso-2-Br-LSD | Report result—typically | 0.02% area |
| | NMT 0.1% area | |

NMT = not more than;
NLT = not less than;
ND = not detected

### Stage 3: Synthesis of 2-Bromolysergic Acid Diethylamide Hemi-L-Tartrate

2-Bromolysergic acid diethylamide (30.2 g, limiting reagent) was charged to a vessel and 2-propanol (420 mL, 13.9 mL/g of 2-Br-LSD) was added, resulting in a brown solution. This solution was transferred via a Supapore PP 1.2 μm in-line filter capsule into a clean flask. A solution of L-tartaric acid (5.7 g, 0.1887 g/g of 2-Br-LSD, 0.5 equiv) in 2-propanol (60 mL, 2 mL/g of 2-Br-LSD) was transferred via a Supapore PP 1.2 μm in-line filter capsule into a dropping funnel set-up on the flask containing the 2-Br-LSD solution. While maintaining the batch temperature at 15° C.-25° C., the tartaric acid solution was added over 25 minutes, resulting in a final batch temperature of 18.8° C. The batch was stirred for 117 minutes at 15° C.-25° C., resulting in a thick white suspension with a final batch temperature of 17.8° C. The mixture was filtered, and the solids were washed with filtered 2-propanol (2×60 mL, 2×2 mL/g of 2-Br-LSD). The cake was pulled dry for ˜30 minutes and dried in vacuo at 65° C.-70° C. to give 27.1 g (75.7% yield; 15.1% from lysergic acid) of off-white solid. The identity was confirmed by comparison of the 1H NMR, MS and FTIR spectra to the corresponding reference spectra. Analysis by HPLC Method 4 showed 99.9% chemical purity with non-detectable LSD, 0.02% iso-2-Br-LSD, and 0.13% total impurities. Any products arising from di- or tri-bromination of lysergic acid in Stage 1 were controlled in the final API (2-bromolysergic acid diethylamide hemi-L-tartrate) as unknowns, with a specification of not more than 0.15% each. In the present batch, they were actually much lower than this specification limit, because total impurities was only 0.13%. L-Tartaric acid content by ¹H NMR: 16.6%. PF₆ content by ¹⁹F NMR: <1500 ppm. Residual solvents by headspace GC: 2-propanol 49 ppm; ethyl acetate 4 ppm; THF <7 ppm; isopropyl acetate not detected; heptane <67 ppm. TMU by HPLC <500 ppm. Residue on ignition: 0.1% w/w. Elemental impurities by ICP-MS: Ni, As, Cd, Co, Hg, Pb, V each <0.1 ppm.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

What is claimed is:

1. A method of synthesizing pharmaceutical grade 2-bromolysergic acid diethylamide (2-Br-LSD), or a pharmaceutically acceptable salt thereof comprising:
a. contacting lysergic acid with bromotrimethylsilane (TMSBr) and dimethylsulfoxide (DMSO), wherein the TMSBr and DMSO are in molar equivalents of about 6 and about 0.9, respectively, to form 2-bromolysergic acid;
b. contacting the 2-bromolysergic acid with diethylamine and an amide coupling reagent, to form 2-Br-LSD;
c. filtering a mixture of the 2-Br-LSD and a solvent;
d. allowing the 2-Br-LSD to precipitate from the filtered mixture;
e. collecting the 2-Br-LSD by filtration;
f. analyzing the collected 2-Br-LSD of (e) for the presence of iso-2-Br-LSD; and
g. (1) if a batch of the collected 2-Br-LSD of (e) meets pre-set specifications that comprise a pre-set specification for iso-2-Br-LSD, then accepting the collected 2-Br-LSD for further processing into pharmaceutical grade 2-Br-LSD, or a pharmaceutically acceptable salt thereof; or
g. (2) if a batch of the collected 2-Br-LSD of (e) fails to meet the pre-set specification for iso-2-Br-LSD, then purifying the collected 2-Br-LSD and repeating (f) and (g), or discarding the collected 2-Br-LSD.

2. The method of claim 1, wherein the pre-set specification for iso-2-Br-LSD is not more than 2%.

3. The method of claim 1, wherein the pre-set specification for iso-2-Br-LSD is not more than 0.4%.

4. The method of claim 3, further comprising at least one of the following pre-set specifications: not more than 0.05% of lysergic acid diethylamide (LSD), not more than 0.5% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

5. The method of claim 3, further comprising at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

6. The method of claim 1, wherein (a) comprises contacting the lysergic acid with TMSBr and DMSO in the presence of a solvent.

7. The method of claim 6, wherein the solvent is tetrahydrofuran (THF).

8. The method of claim 1, wherein (b) comprises contacting the 2-bromolysergic acid with diethylamine and an amide coupling reagent in the presence of an organic solvent.

9. The method of claim 8, wherein the organic solvent is THF.

10. The method of claim 1, wherein the amide coupling reagent is propylphosphonic anhydride (T3P), 2-(1H-benzotriazol-1-yl)-1,1,3,3-tetramethyluronium hexafluorophosphate (Hexafluorophosphate Benzotriazole Tetramethyl Uronium, or HBTU), 1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium, or HATU), 1,1′-Carbonyldiimidazole (CDI), or acetic anhydride.

11. The method of claim 10, wherein the amide coupling reagent is T3P.

12. The method of claim 10, wherein the amide coupling reagent is HBTU.

13. The method of claim 1, wherein the solvent of (c) comprises an organic solvent.

14. The method of claim 13, wherein the organic solvent comprises isopropyl acetate or ethyl acetate.

15. The method of claim 13, wherein the solvent of (c) further comprises water in an amount of less than about 3.5%.

16. The method of claim 13, wherein water is added to the filtered mixture of (c) in an amount of less than about 3.5%.

17. The method of claim 16, wherein (c) is performed at or below about 20° C. to about 25° C., the filtered mixture from (c) is heated to ensure complete dissolution prior to the addition of water, and (e) is performed at or below about 20° C. to about 25° C.

18. The method of claim 1, wherein (c) is performed at a temperature above about 20° C. to about 25° C. and (d) is performed at or below about 20° C. to about 25° C.

19. The method of claim 18, wherein (c) is performed at about 30° C.-50° C.

20. The method of claim 1, wherein the purifying in (g2) comprises column chromatography.

21. The method of claim 1, wherein the purifying in (g2) comprises HPLC.

22. A batch of pharmaceutical grade 2-Br-LSD, or a pharmaceutically acceptable salt thereof, that meets pre-set specifications that comprise a pre-set specification for iso-2-Br-LSD, wherein the pre-set specification for iso-2-Br-LSD is not more than 0.4%.

23. The batch of claim 22, further comprising at least one of the following pre-set specifications: not more than 0.05% of LSD, not more than 0.5% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

24. The batch of claim 22, further comprising at least one of the following pre-set specifications: not more than 0.05% of LSD, not more than 0.05% of iso-LSD, not more than 0.15% each of di-bromo-LSD species comprising 2,12-dibromo-LSD, 2,13-dibromo-LSD, and 2,14-dibromo-LSD, and not more than 0.15% each of tri-bromo-LSD species comprising 2,12,13-tribromo-LSD, 2,12,14-tribromo-LSD, and 2,13,14-tribromo-LSD.

25. A pharmaceutical composition, comprising the batch of claim 22, or a portion thereof, and a pharmaceutically acceptable carrier.
